(19) United States
(12) Patent Application Publication
(10) Pub. No.: US 2005/0070854 A1
(43) Pub. Date: Mar. 31, 2005

(54) Syringe

(76) Inventor: Alastair Douglas Wright, Edinburgh (GB)

Correspondence Address: BAKER & DANIELS;111 E. WAYNE STREET, SUITE 800, FORT WAYNE, IN, 46802, US

(21) Appl. No.: 10/670,643

(22) Filed: Sep. 25, 2003

(51) Int. Cl. A61M 5/32

(52) U.S. Cl. 604/197; 604/110

(57) ABSTRACT

A syringe (1) comprises a barrel (2) having a needle (23) engaged in a glass hub (19). The glass hub (19) is retractable under the action of a spring (30) towards the trailing end of the syringe. The glass hub (19) is restrained against movement towards the trailing end by a crown (10) which is removably attached to the glass hub (19). A plunger (36) engages the crown (10), removing it from the glass hub (19) and allowing retraction of the needle (23). A core (45), removably attached to the end of the plunger by a resiliently deformable member, is then removed from the plunger, allowing the glass hub (19) and the needle (23) to be retracted into the plunger under the action of the spring (30).

### FIELD OF THE INVENTION

The present relates to a syringe and, in particular, a syringe having a retractable needle. The present invention also relates to a collar attachable to a syringe and a needle unit for a syringe.

### DESCRIPTION OF THE PRIOR ART

It has long been known to administer medicaments and the like transcutaneously to an individual by using a syringe having a hypodermic needle. The syringe has a plunger slidably received in a barrel and, when the plunger is depressed, a liquid medicament is expelled from the hypodermic needle.

The problem with such syringes is that the hypodermic needle remains an extremely sharp object after use which can be dangerous both because of the physical damage that such a sharp object can cause and because, once the needle has been inserted into an individual, it may transmit infections that the individual was carrying.

One solution to this problem is to place a cap, or sheath, over the needle after it has been used so that the needle is enclosed. However, the problem with doing this is that, in practice, it is quite difficult to place such a cap over the end of the syringe. The needle is very thin and an individual may accidentally prick himself with the needle when attempting to put the cap in place. Furthermore, the cap may inadvertently fall off the syringe, once it has been fitted.

Another problem with this type of syringe is that, amongst certain groups of people (e.g. illegal drug users) there is a temptation to use syringes repeatedly, with the same needle, which results in a very high risk of cross-infection between members of the group.

Accordingly, a solution to the problems encountered with such syringes is a syringe with a retractable needle. WO-A-96/05879 discloses such a syringe wherein the needle is spring-loaded so that, when the plunger in the syringe reaches the end of its stroke, a frangible section in a leading end of the plunger is broken off.

Furthermore, a restraining member, which holds the needle in place, against the force of the spring is dislodged so that the spring forces the needle through the leading end of the plunger and into the hollow interior of the plunger. Thus, once all (or almost all) of the liquid has been expelled from the barrel of the syringe, the needle is automatically retracted into the interior of the plunger, which lies within the barrel of the syringe. Thus, once the needle has been retracted, it is no longer a danger to others and the syringe may not be reused.

WO-A-02/087669 also discloses a syringe having a retractable needle. A hub, which is attached to the needle, and the restraining member are formed as plastic mouldings that engage with one another and which are disengaged at or towards the end of the plunger's stroke.

Syringes are normally supplied empty, so that an individual may choose a particular medicament or the like to draw into the syringe before administering it. However, for the sake of convenience, it is also known to supply pre-filled syringes, especially of the most commonly required medicaments such as insulin.

The problem that arises with pre-filled syringes is that, while normally the majority of the components of a syringe are made from plastics materials, this is not possible with pre-filled syringes because the syringes may be stored for relatively long periods of time in which the enclosed medicament is in contact with the plastics materials. Some medicaments are not stable when they are in long term contact with a plastics material and, in practice, it is often difficult to determine whether or not a particular medicament is stable when in contact with a particular plastics material (or, at least, it is difficult to obtain the appropriate medical authorisation to store the medicaments in a plastics material). Therefore, it is almost always necessary for the components of a pre-filled syringe that come into contact with the medicament to be made either from glass or from rubber but not from a plastics material. This presents a problem for producing a pre-filled syringe with a retractable needle because the syringes with retractable needles disclosed in the prior art (for example WO-A-96/05879 and WO-A-02/087669) rely on components which are made from a plastics material, in particular the frangible or disengagable components disclosed therein.

Another problem with the syringes having retractable needle mechanisms known in the prior art is that the needle retraction mechanisms may inadvertently be activated prematurely. For example, during transport of the syringe, the plunger of the syringe may be depressed with sufficient force to activate the retraction mechanism. Of course, once the needle has been retracted, the syringe cannot be used and is wasted.

Another problem that arises, generally, in the field of syringes is that, during use, the plunger is inadvertently drawn so far along the barrel of the syringe that it actually falls completely out of the end of the barrel. One instance where this can occur is when the syringe is being filled (although this will not, of course, occur if it is a pre-filled syringe). Another instance is while the medicament is administered to an individual. One technique for use of a syringe is, having inserted the hypodermic needle into a blood vessel of the individual, to draw a little blood from the individual by withdrawing the plunger slightly. This allows the individual performing the injection to be certain that the hypodermic needle has been inserted into a blood vessel. In either of these two instances, the plunger may, inadvertently, be withdrawn too far and, if so, the contents of the syringe is usually lost and the procedure must be repeated.

It is known in the art to provide a stop at the end of the barrel of the syringe in order physically to prevent the plunger from being withdrawn too far. The problem, however, with pre-filled syringes is that, as has previously been explained, the barrel of the pre-filled syringe must be made from glass. There are limitations to the complexity that can be introduced into the shape of a glass barrel and therefore it is not usually possible for the glass barrel to have any form of stop to prevent the withdrawal of the plunger. Therefore, pre-filled syringes still suffer from the problem of the plunger being inadvertently withdrawn too far.

### SUMMARY OF THE INVENTION

The present invention seeks to alleviate one or more of the above problems.

According to one aspect of the present invention, there is provided a syringe comprising:
a barrel having leading and trailing ends;
a hollow bore needle extending from the leading end of the barrel and carried by a needle hub;
a resilient member connected between the barrel and the needle hub so as to urge the needle hub and the needle toward the trailing end of the barrel;
a crown removably attached to the needle hub and the barrel and preventing movement of the needle hub under the influence of the resilient member; and
a plunger slidably moveable within the barrel;
   wherein, when the plunger is moved along the barrel towards the leading end, the plunger engages the crown so as to release the attachment between the crown and the needle hub allowing the needle hub, and the needle to be retracted by the resilient member.

Conveniently, the plunger carries a removable core at its leading end leading to a hollow interior, the hollow interior being sized to receive the needle and the needle hub such that, when the plunger is moved along the barrel towards the leading end, the needle hub engages the core so as to release the attachment between the core and the plunger allowing the needle hub, and the needle to be retracted into the hollow interior of the plunger by the resilient member.

Preferably, the core is removably attached to the plunger via a resiliently deformable member.

Advantageously, the crown is removably attached to the needle hub via a resiliently deformable member.

Conveniently, the resiliently deformable member attaching the crown to the needle hub forms a seal around the crown, between the needle hub and the barrel.

Preferably, the resiliently deformable member attaching the core to the plunger forms a seal around the end of the plunger, between the removable core and the barrel.

Advantageously, the two resiliently deformable members are made from rubber and the barrel, the needle hub and the removable core are made from glass.

According to another aspect of the present invention, there is provided a needle unit for a syringe, the needle unit comprising:
a needle connected to a retraction mechanism for retracting the needle; and
a removable sheath for enclosing the needle,
   wherein the sheath engages the retraction mechanism when it encloses the needle to prevent retraction of the needle prior to removal of the sheath.

According to a further aspect of the present invention, there is provided a syringe comprising such a needle unit.

According to another aspect of the present invention, there is provided a collar for attachment to a syringe having a barrel and a retractable plunger extending from one end of the barrel the collar comprising:
means to grip said end of the barrel; and
at least one stop member located so that, when the collar is attached to the barrel, the stop member projects over said end of the barrel such that, when the plunger is retracted from the barrel, a portion of the plunger impinges on the stop, preventing removal of the plunger from the barrel.

Conveniently, the collar is made from a plastics material.

According to a further aspect of the present invention, there is provided a syringe comprising: a barrel; a retractable plunger extending from one end of the barrel; and a collar as described above.

Preferably, the barrel is made from glass.

Throughout this specification, it is to be understood that the term “leading end” refers to the end of the syringe, or a component of the syringe, towards the needle whereas the term “trailing end” refers to the end of the syringe, or a component of the syringe, towards the plunger.

### BRIEF DESCRIPTION OF THE DRAWINGS

In order that the present invention may be more readily understood, and so the further features thereof may be appreciated, embodiments of the invention will now be described, by way of example, with reference to the accompanying drawings in which:

FIG. 1A is an axial cross-sectional view of a syringe in accordance with an embodiment of the present invention;

FIG. 1B is an enlarged view of the leading end of the syringe shown in FIG. 1A;

FIG. 1C is an enlargement of the needle unit of the syringe shown in FIG. 1A;

FIG. 1D is an enlargement of a section of the plunger of the syringe shown in FIG. 1A;

FIG. 1E is an enlargement of the trailing end of the barrel of the syringe shown in FIG. 1A;

FIG. 2 is a perspective view of a needle unit of a syringe in accordance with an embodiment of the present invention, with part cut away;

FIG. 3 is a perspective view of a section of a plunger of a syringe in accordance with an embodiment of the present invention, with part cut away; and

FIG. 4 is a perspective view of a section of the trailing end of a syringe in accordance with an embodiment of the present invention, with part cut away.

### DESCRIPTION OF THE PREFERRED EMBODIMENTS

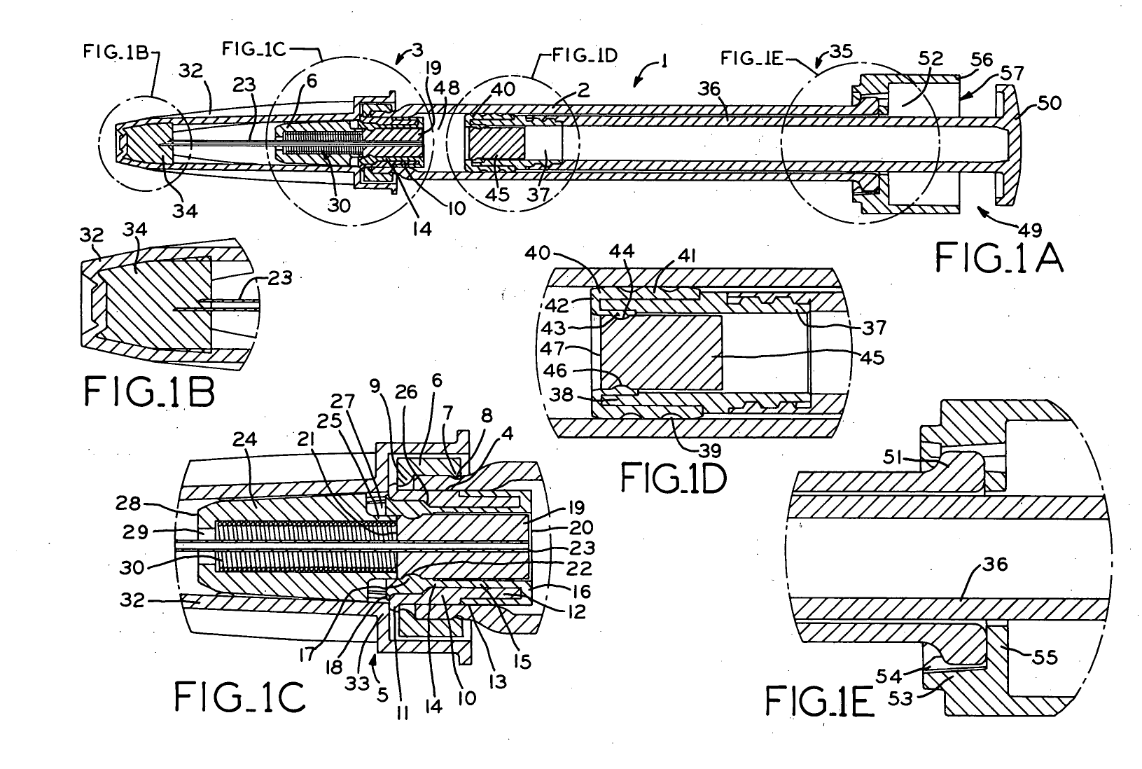

Referring to FIG. 1 (and the enlargements FIGS. 1B, 1C, 1D and 1E), a syringe 1 is shown in axial cross-section. The syringe 1 comprises a hollow glass barrel 2 which is substantially cylindrical. At its leading end 3, the barrel 2 narrows to a neck 4 in which is received a needle unit 5 which is held in place by a generally annular end cap 6. The end cap 6 sits around the exterior of the neck 4 having an inwardly projecting ridge 7 which sits in an annular groove 8 in the exterior of the neck 4. The end cap 6 is also provided with a leg 9 which protrudes over the leading end 3 of the barrel so that the end of the leg lies flush with the interior surface of the barrel 2. Thus the end cap 6 holds the needle unit 5 in place as will be described in greater detail below.

The needle unit 5, which is shown in detail in FIG. 1C, comprises a generally annular crown 10 made from a plastics material, which abuts the interior of the neck 4 of the barrel 2. At its leading end, the crown 10 has a radially outwardly extending leg 11 which sits over the leg 9 of the end cap 6. Thus the crown 10 is prevented from moving towards the trailing end of the barrel 2. The end of cap 6 has four cut outs 31, equally spaced about its circumference, which expose the leg 11 of the crown 10. Towards the trailing end of the crown 10 is a step leading to a finger 12. The finger 12 sits radially inwardly of the neck 4 of the barrel 2 and thus an annular channel 13 exists between the finger 12 and the neck 4.

Also comprised in the needle unit 5 is a generally annular needle unit seal 14 made from rubber. The needle unit seal 14 has a central section 15 which sits radially inwardly of the crown 10. The central section is of the needle unit seal 14 and the adjacent section of the crown 10 are shaped so as to mutually engage one another about a ramped step 26 which is formed such that the needle unit seal 14 is thicker and the crown 10 is thinner in the leading direction from the ramped step 26. Thus the engagement of the needle unit seal 14 and the crown 10 at the ramped step 26 prevents the movement of the crown in a leading direction and the movement of the needle unit seal 14 in a trailing direction.

At its trailing end, the needle unit seal 14 has a U-shaped section 16 which extends in a trailing direction before extending around the finger 12 of the crown 10 and then extending inwardly to sit in the annular channel 13. Thus, the U-shaped section 16 effectively encapsulates the trailing end of the crown 10. At its leading end, the needle unit seal 14 has a radially inwardly extending ridge 17, which will be described in greater detail below and, at its tip, a radially outwardly extending flange 18 which sits over the leg 11 of the crown 10.

The crown 10 and the needle unit seal 14 can be produced together by a two shot injection moulding process.

Also forming part of the needle unit 5 and located radially inwardly of the needle unit seal 14 is a substantially cylindrical glass hub 19 which has an axial bore therethrough. The trailing end 20 of the glass hub 19 lies flush with the end of the U-shaped section 16 of the needle unit seal 14. Around the exterior of the glass hub 19, towards its leading end 21, there is provided an annular groove 22, in which the ridge 17 of the needle unit seal 14 is received. A hollow bore needle 23 extends through the axial bore of the glass hub 19 and is attached thereto.

It is to be appreciated that the needle unit seal 14 provides a seal between the barrel 2 and the glass hub 19, preventing the passage of liquid therebetween.

Adjacent the leading end 21 of the glass hub 19 the end cap 6 forms a hollow, frustro-conical nose 24. At the trailing end of the nose 24 is provided a narrow ring 25 which abuts the leading end 21 of the glass hub 19 and spaces the nose 24 from the flange 18 of the needle unit seal 14, thus leaving an annular chamber 27 therebetween. There is an aperture 29 in the leading end 28 of the nose 24. The needle 23 extends through the nose 24 and exits the nose 24 through the aperture 29.

Received within the hollow interior of the nose 24 is a coiled spring 30 which extends axially, around the needle 23, from the leading end 21 of the glass hub 19 to the leading end 28 of the nose 24. The spring 30 is under compression and therefore presses against the leading end 28 of the nose 24 to urge the hub 19 (together with the needle 23 which is attached thereto) in a trailing direction. However, the engagement of the ridge 17 of the needle unit seal 14 in the groove 22 of the hub 19 prevents the hub 19 from moving in a trailing direction. In this regard, it is to be appreciated that the needle unit seal 14 is prevented from moving in a trailing direction because of its engagement with the crown 10 and, in particular, the engagement of the flange 18 over the leg 11 of the crown 10. It is also to be appreciated that the crown 10 is prevented from moving in a trailing direction because of the engagement of the leg 11 of the crown 10 over the leg 9 of the end cap 6.

Encasing the needle unit 5 of the syringe 1 is a cap, or sheath 32 which is removably seated around the end cap 6, including the nose 24 thereof. The cap 32 has four radially inwardly extending ribs 33 which are equally spaced about the interior circumference thereof. The ribs 33 are received in the cut outs 31 of the end cap 6 and sit against the leading edge of the leg 11 of the crown 10. Thus, while the cap 32 is in place, the ribs 33 prevent the movement of the crown 10 in a leading direction.

A rubber bung 34 is provided in the inside of the tip of the cap 32 as is shown in FIG. 1B. The tip of the needle 23 is removably received in the rubber bung 34. The friction between the needle 23 and the rubber bung 34 prevents accidental removal of the cap 32.

Having thus described the needle unit 5 of the syringe 1, the other components of the syringe 1 will now be described.

Received in the trailing end 35 of the barrel 2 is a hollow plunger 36 whose exterior circumference is slightly smaller than the interior circumference of the barrel 2. As is most clearly shown in FIG. 1D, at the leading end of the plunger 36 a screw thread on the interior of the plunger 36 engages with an actuator element 37 which is generally tubular. The leading end of the actuator element 37 terminates in a narrow annular finger 38 whose outer circumference is less than the inner circumference of the barrel 2. Thus an annular cavity 39 exists between the finger 38 and the interior of the barrel 2. Sitting in the cavity 39 is a plunger seal 40 which is of approximately “J” shaped cross-section and is made from rubber. Thus the plunger seal 40 has a body 41 which sits in the annular cavity 39 between the barrel 2 and the narrow annular finger 38. The body 41 leads to an end section 42 which sits over the leading end of the actuator element 37 and then leads to an inner section 43 on the inner surface of the actuator element 37. Thus the plunger seal 40 encapsulates the end of the actuator element 37. The inner section 43 of the plunger seal 40 has a radially inwardly projecting ridge 44 which will be described in further detail below.

The actuator element 37 and the plunger seal 40 can be produced together by a two hot injection moulding process.

Located within the hollow interior of the actuator element 37 is a substantially cylindrical glass core 45 having a diameter greater than the glass hub 19. The glass core 45 is held in place within the actuator element 37 by the engagement of the inwardly projecting ridge 44 of the plunger seal 40 in an annular groove 46 located in the exterior of the glass core 45, towards its leading end 47. The leading end 47 of the glass core 45 is recessed slightly compared with the end section 42 of the plunger seal 40 which thus protrudes around the leading end 47 of the glass core 45.

The plunger seal 40 fits tightly against the interior of the barrel 2 and thus has a greater circumference than the plunger 36. Accordingly, the plunger seal 40 provides a seal between the glass core 45 and the interior of the barrel 2. Thus, while the plunger seal 40 allows the plunger 36 to be moved slidably within the barrel 2, it also prevents the escape of a liquid, such as a medicament, contained within a storage chamber 48 which is defined by the barrel 2, the plunger 36 and the needle unit 5.

As is shown in FIG. 1E, at the trailing end 49 of the plunger 36 is provided a mushroom-shaped grip 50 having a circumference greater than the circumference of the barrel 2.

The trailing end of the barrel 2 has an enlarged radially outwardly projecting rim 51. A plastic collar 52 is clipped onto the enlarged rim 51 by means of an annular claw 53. The claw 53 comprises a leading leg 54 which sits on the leading side of the rim 51 and a trailing leg 55 which sits on the trailing side of the rim, at the very end of the trailing end of the barrel 2. The trailing leg 55 extends radially inwardly, beyond the interior surface of the barrel 2, but not so far as to contact the outer surface of the plunger 36.

The trailing end of the collar 52 comprises an annular wall 56 defining a cylindrical recess 57 which is sized to receive the plunger 36 and, more particularly, the grip 50. The inner circumference of the annular wall 56 is only very slightly larger than the circumference of the mushroom-shaped grip 50 and thus is sized to receive the grip with a snug fit.

In use, the syringe 1 is manufactured so as to have a liquid contained within the storage chamber 48. The liquid is typically a medicament such as insulin. Thus the syringe 1 is a pre-filled syringe. It is to be appreciated that the components of the syringe 1 which are in contact with the liquid contained in the storage compartment (namely the barrel 2, the needle unit seal 14, the glass hub 19, the plunger seal and the glass core 45) are all made from materials with which the liquid may safely come into contact without being degraded. Thus, the pre-filled syringe may be stored for long periods of time without the liquid being affected.

Even though the barrel 2 is made from glass, the plastic collar 52 is still provided, clipped to the barrel 2, in order to prevent over-withdrawal of the plunger 36 and re-use of the syringe 1 as is explained in greater detail below.

In order that the storage chamber 48 is of suitable size so that a sufficient quantity of liquid is stored therein, the plunger 36 is in a withdrawn position, most of the way out of the barrel 2. Thus the plunger 36 extends out of the end of the barrel 2 and the collar 52 and the grip 50 sits remote from the collar 52.

It is also to be noted that, when stored, the sheath or cap 32 is held in place by its frictional engagement with the end cap 6 and by the engagement of the needle 23 in the rubber bung 34. While the cap 32 is held in this position, the rib inwardly extending ribs 33 on the cap 32 engage the crown 10 and prevent the movement of the crown 10 in a leading direction.

When it is desired to administer the liquid contained in the syringe to an individual, the cap 32 is removed from the leading end of the syringe 1 by pulling the two components apart axially. As will be appreciated, once the cap 32 has been removed, the crown 10 is no longer prevented from moving towards the leading end of the syringe 1 by the inwardly extending ribs 33 on the cap 32 although it is still prevented from such movement by the engagement with the needle unit seal 14 at the ramped step 26 and the flange 18.

The needle 23 is then inserted into the blood vessel of the individual as is usual with hypodermic syringes. If desired, the plunger 36 may then be slid slightly outwardly of the barrel 2 by pulling on the grip 50. In this way, a small amount of blood is drawn into the needle 23 and the storage chamber 48 which can be seen and which thus confirms that the needle 23 is, indeed, located in a blood vessel. During the process of sliding the plunger 36 outwardly of the barrel 2, it is not possible for the plunger 36 to be completely removed from the barrel 2 because the trailing leg 55 of the claw 53 of the collar 52 projects over the trailing end of the barrel 2 and impinges upon the plunger seal 40 as the plunger 36 is drawn out of the barrel 2. When the plunger seal 40 impinges upon the trailing leg 55, further movement of the plunger 36 in a trailing direction is prevented.

Whether or not there is any withdrawal of the plunger 36, the next step in administering the liquid is for the plunger 36 to be slid along the barrel 2 towards the leading end of the barrel 2. This is achieved by depressing the grip 50. The liquid in the chamber 48 is thus pressurised and, when sufficient pressure is reached, the liquid is expelled via the needle 23 out of its tip and into the blood vessel of the individual, as is normal for hypodermic syringes known in the art.

As the plunger 36 continues to be depressed, the leading end 42 of the plunger seal 40 comes into contact with the trailing end 16 of the needle unit seal 14. It is to be noted that the leading end 42 of the second rubber seal 40 is similarly sized and aligned with the trailing U-shaped section 16 of the needle unit seal 14. Thus, when the respective seals 14, 40 meet, the user of the syringe 1 can feel an appreciable increase in the force required to depress the plunger 36. However, if the user continues to depress the plunger 36, then the plunger seal 40 pushes the needle unit seal 14 towards the leading end of the syringe 1 with the inwardly extending ridge 17 of the needle unit seal 14 being radially outwardly compressed and deflected by virtue of its resilient nature, and thus pushed out of the annular groove 22 in the glass hub 19. Because of the engagement of the needle unit seal 14 about the finger 12 of the crown 10, the crown 10 is also pushed in a leading direction. Thus the flange 18 of the needle unit 14 is pushed into the annular chamber 27 as the needle unit seal 14 slides axially against the glass hub 19 and the crown 10 slides axially against the neck 4 of the barrel 2.

It is to be appreciated that, as the ridge 17 of the needle unit seal 14 leaves the groove 22 in the glass hub 19, the urging of the glass hub 19, towards the trailing end of the syringe 1, by the spring 30 is no longer restrained by the crown 10 and the needle unit seal 14.

However, as has previously been explained, the leading end 47 of the glass core 45 is recessed compared with the plunger seal 40. As the plunger seal 40 pushes the needle unit seal 14 towards the leading end of the syringe 1, the leading end 47 of the glass core 45 comes into contact with the trailing end 20 of the glass hub 19. Accordingly, the glass core 45 prevents the movement of the glass hub 19 towards the trailing end of the syringe 1 under the action of the spring 30.

As the user of the syringe 1 continues to depress the plunger 36, the leading end 47 of the glass core 45 presses against the trailing end 20 of the glass hub 19. The glass hub 19 is effectively prevented from moving towards the leading end of the syringe 1 because it rests against the ring 25 of the end cap 6 which is firmly attached to the neck 4 of the barrel 2. Thus a force is applied to the glass core 45 in a trailing direction. Once a predetermined threshold of force has been reached, the inwardly projecting ridge 44 of the plunger seal 40 is radially outwardly compressed and deflected by virtue of the resilient nature of the ridge 44, and pushed out of the groove 46 in the glass core 45. Once the ridge 44 of the plunger seal 40 has been pushed out of the groove 46 in the glass core 45, the spring 30 is able to act to urge the glass hub 19, the needle 23, to which it is attached, and the glass core 45 towards the trailing end of the syringe 1 which is thus enclosed in the hollow interior of the plunger 36. It is to be noted that since the glass hub 19 is of slightly smaller diameter than the glass core 45, the glass hub 19 does not impinge upon the ridge 44 of the plunger seal 40. Accordingly, the needle 23 is retracted inwardly and is encased within the barrel 2 and the plunger 36 once the plunger is fully depressed so that the syringe 1 cannot be reused and, furthermore, the needle 23 is safely enclosed within the syringe 1. Thus the needle 23 is no longer a scratch or puncture hazard.

As the plunger 36 is depressed, the grip 50 is received within the cylindrical recess 57 of the collar 52. The wall 56 which thus surrounds the grip 50 is relatively tight fitting. The fit is such that the grip 50 may readily continue to be depressed but it is very difficult, if not impossible, for the grip 50 to be pulled outwardly since there is no surface on which a user may get a purchase on the grip 50. Thus, the user (or, indeed, any other individual) is prevented from retracting the plunger 36, once the grip 50 has passed the end of the wall 56. Consequently, it is not possible for the plunger 36 to be retracted and the syringe reused.

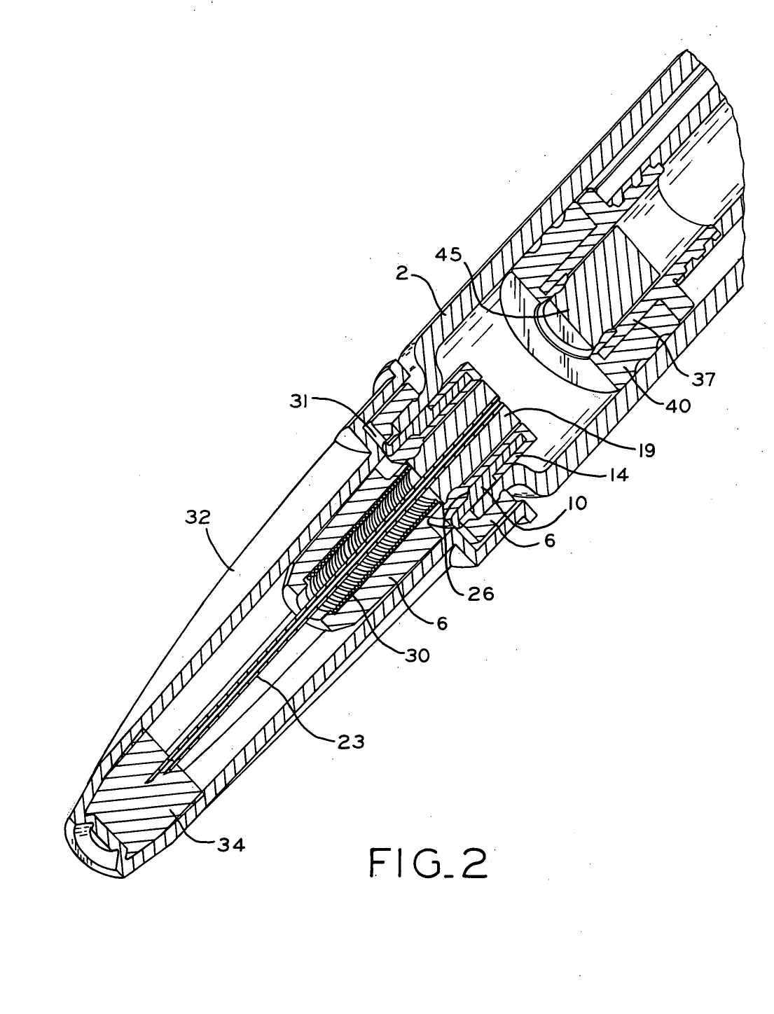

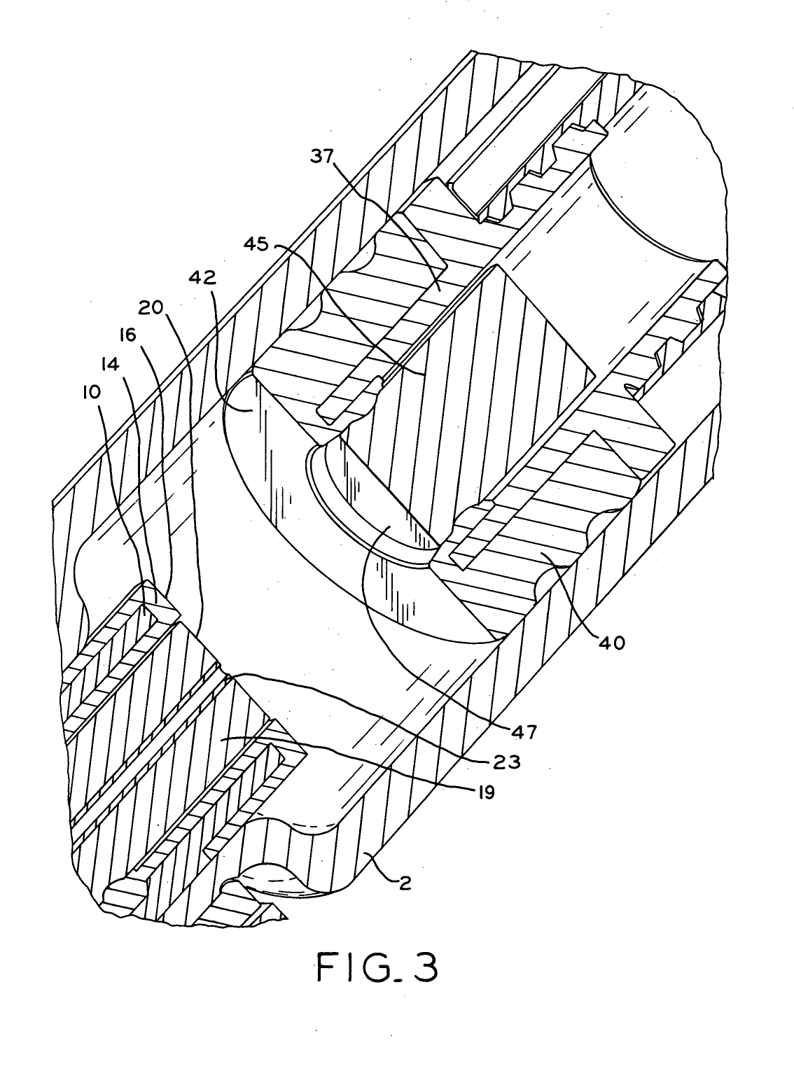

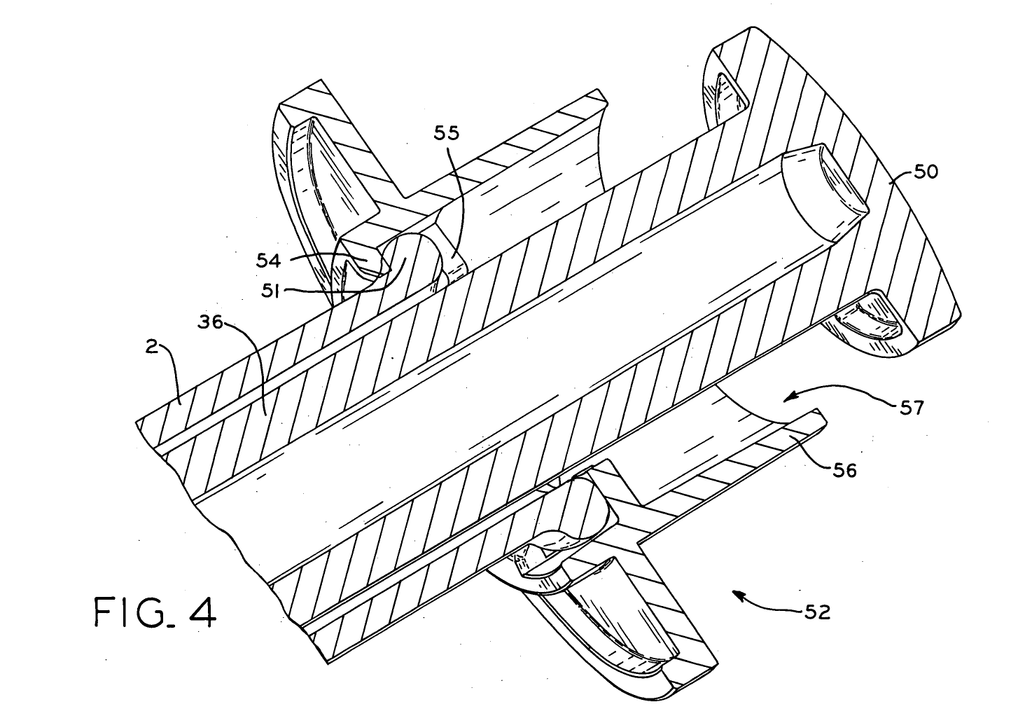

In FIGS. 2, 3 and 4, portions of a further embodiment of the present invention are shown. The further embodiment is only very slightly different from the above described embodiment and the corresponding components are given the same reference numbers. The functioning of the further embodiment is substantially the same as the above described embodiment.

1. A syringe comprising:
a barrel having leading and trailing ends;
a hollow bore needle extending from the leading end of the barrel and carried by a needle hub;
a resilient member connected between the barrel and the needle hub so as to urge the needle hub and the needle toward the trailing end of the barrel;
a crown removably attached to the needle hub and the barrel and preventing movement of the needle hub under the influence of the resilient member; and
a plunger slidably moveable within the barrel; the plunger carrying a removable core at its leading end leading to a hollow interior, the hollow interior being sized to receive the needle and the needle hub;
such that when the plunger is moved along the barrel towards the leading end, the plunger engages the crown and the needle hub engages the core so as to release the attachment between the crown and the needle hub and between the core and the plunger allowing the needle hub, and the needle to be retracted into the hollow interior of the plunger by the resilient member, wherein the core is removably attached to the plunger via a first resiliently deformable member.

2. A syringe according to claim 1, wherein the crown is removably attached to the needle hub via a second resiliently deformable member.

3. A syringe according to claim 2, wherein the second resiliently deformable member forms a seal around the crown, between the needle hub and the barrel.

4. A syringe according to claim 3, wherein the first resiliently deformable member forms a seal around the end of the plunger, between the removable core and the barrel.

5. A syringe according to claim 4, wherein the first and second resiliently deformable members are made from rubber and the barrel, the needle hub and the removable core are made from glass.

6. A needle unit for a syringe, the needle unit comprising:
a needle connected to a retraction mechanism for retracting the needle; and
a removable sheath for enclosing the needle,
wherein the sheath engages the retraction mechanism when it encloses the needle to prevent retraction of the needle prior to removal of the sheath.

7. A syringe comprising a needle unit according to claim 6.

8. A collar for attachment to a syringe having a barrel and a retractable plunger extending from one end of the barrel the collar comprising:
means to grip said end of the barrel; and
at least one stop member located so that, when the collar is attached to the barrel, the stop member projects over said end of the barrel such that, when the plunger is retracted from the barrel, a portion of the plunger impinges on the stop, preventing removal of the plunger from the barrel.

9. A collar according to claim 8 wherein the collar is made from a plastics material.

10. A syringe comprising: a barrel; a retractable plunger extending from one end of the barrel; and a collar according to claim 8.

11. A syringe according to claim 10 wherein the barrel is made from glass.
